(19) United States
(12) Patent Application Publication
(10) Pub. No.: US 2025/0099643 A1
(43) Pub. Date: Mar. 27, 2025

(54) DEVICE FOR DISINFECTING AN AIR FLOW VIA UV-C RADIATION AND ASSISTED VENTILATION SYSTEM COMPRISING SUCH A DEVICE

(71) Applicant: ISTITUTO NAZIONALE DI ASTROFISICA, ROMA (IT)

(72) Inventors: Matteo LOMBINI, Roma (IT); Alessio ZANUTTA, Roma (IT); Andrea BIANCO, Roma (IT); Luigi LESSIO, Roma (IT); Fausto CORTECCHIA, Roma (IT); Giuseppe MALAGUTI, Roma (IT); Giovanni PARESCHI, Roma (IT); Emiliano DIOLAITI, Roma (IT); Adriano DE ROSA, Roma (IT)

(21) Appl. No.: 18/558,946

(22) PCT Filed: May 6, 2022
(86) PCT No.: PCT/IB2022/054193

§ 371 Date: Nov. 3, 2023

(30) Foreign Application Priority Data
May 7, 2021 (IT) 102021000011783

(51) Int. Cl. A61L 9/20; A61M 16/06

(52) U.S. Cl. CPC A61L 9/20; A61M 16/0627; A61L 2209/12; A61L 2209/16

(57) ABSTRACT

A device for disinfecting an air flow, comprising a hollow body having an axis A, a side wall and two base walls provided with internal reflective surfaces, an inlet opening and an outlet opening formed in the respective base walls and at least one source of UV-C radiation disposed within the hollow body, and a helical-shaped deflector axially housed in the hollow body; wherein the hollow body is cylindrical and an outer edge of the deflector cooperates substantially sealingly with the inner surface of the side wall of the hollow body so as to define with said side wall a helical conduit traversed by the entire flow. The device may be used in assisted ventilation systems to disinfect the air exhaled by a patient.

### CROSS-REFERENCE TO RELATED APPLICATIONS

This patent application is related to Italian Patent Application No. 102021000011783 filed on May 7, 2021, the entire disclosure of which is incorporated herein by reference.

### TECHNICAL FIELD OF THE INVENTION

The present invention relates to a device for disinfecting an air flow via UV-C radiation. The invention is preferably applied to the disinfection of air exhaled by a patient undergoing non-invasive assisted ventilation.

### STATE OF THE ART

In this emergency period caused by the Covid-19 pandemic, the disinfection of environments is taking on a key role due to high infectivity. Proven techniques with germicides, UV radiation, etc. may be used for environmental disinfection.

The treatment of the air coming directly from a patient to whom a breathing aid has been applied, for example, in hospital wards for the sub-acute patients, is more critical. In these cases, the air is not always treated before being reintroduced into the environment and the only solution to protect already fragile patients and healthcare workers from exposure to pathogens is to provide all individuals with personal protective equipment (PPE). This involves non-negligible risks in the event of improper use of PPE and disposal thereof. Even when the air exhaled by the patient is treated before being reintroduced into the environment, e.g. by means of electrostatic or membrane filters, the filters have to be changed after a few hours of service, with considerable handling and disposal costs, and with the further difficulty of following strict protocols to maintain the required level of hygiene.

In order to overcome the critical issues associated with the use of filters to treat the air exhaled by a patient undergoing non-invasive oxygen therapy, UV disinfection devices have been developed, in particular UV systems that emit radiation belonging to the UV-C band (between 250 and 280 nm), through which the air flow passes. However, in order to achieve adequate levels of air disinfection, such UV systems require a radiation dosage of several mJ/cm2 (varying depending on the micro-organism to be inactivated), which may be achieved by using relatively high power sources and/or a relatively long residence time of the air in a limited area subjected to radiation.

It derives that the known systems have considerable application limitations; in particular, they are not suitable for implementing compact, portable and/or easily applicable high-flow assisted ventilation solutions.

WO 92/20974 A1 discloses a device for disinfecting a turbulent flow of air at a high flow-rate in a conduit of an environment conditioning system using UV radiation. The device comprises an inlet deflector provided with a plurality of openings and adapted to make the air flow uniform, and a plurality of longitudinal UV lamps around which helical deflectors are arranged with the purpose of creating a rotating and essentially laminar flow of air around the lamps.

The aforementioned device cannot be used effectively in assisted ventilation systems due to its large size; moreover, as the flow in such systems is already essentially laminar, the aforesaid device would be substantially ineffective.

### Subject and Summary of the Invention

Aim of the present invention is to realise a device for disinfecting an air flow that is free of the drawbacks of the known and above specified devices.

In particular, aim of the present invention is to realise a compact, portable device for disinfecting an air flow exhaled by a patient undergoing non-invasive assisted ventilation.

The aforesaid aim is achieved by a device according to claim 1.

The present invention also relates to an assisted ventilation system according to claim 12.

The invention enables to more effectively inactivate pathogenic micro-organisms in the air exhaled by the patient by means of UV-C radiation through the introduction of a forced path with a helical trajectory that ensures a relatively long residence time of the air in the device.

The use of LEDs as UV radiation sources allows to combine high luminous efficiency, small size, robustness and long life.

This makes it possible to realise a compact device that can be advantageously integrated into existing assisted ventilation systems.

### BRIEF DESCRIPTION OF THE DRAWINGS

For a better understanding of the present invention, some preferred embodiments are described hereinafter with reference to the accompanying drawings, wherein:

FIG. 1 is a perspective and schematic view of a disinfection device according to the invention;

FIG. 2 is a diagram illustrating an embodiment of an assisted ventilation system provided with a device according to the present invention; and

FIG. 3 is a perspective view of a second embodiment of a disinfection device according to the present invention.

### DETAILED DESCRIPTION OF PREFERRED EMBODIMENTS OF THE Invention

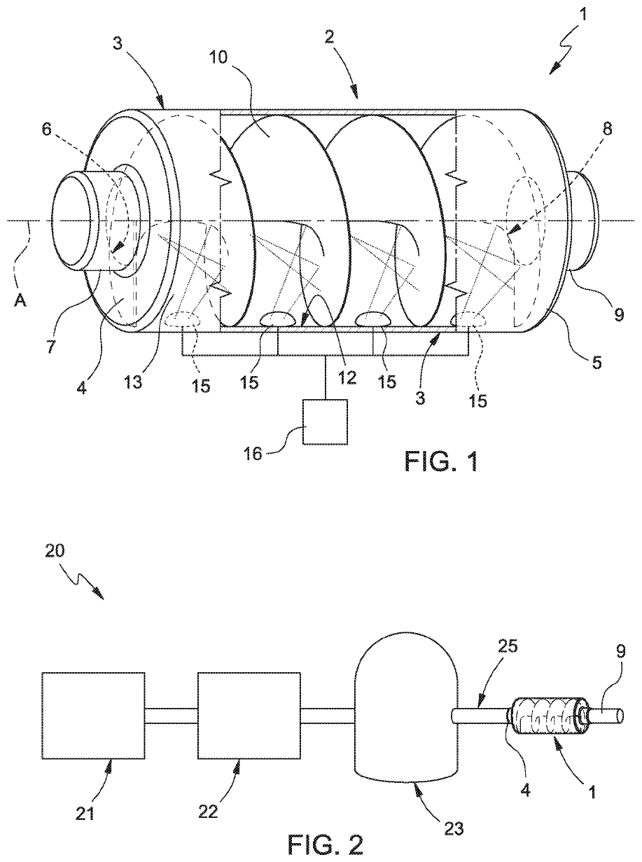

With reference to FIG. 1, an embodiment of a disinfection device 1 according to the present invention is schematically illustrated.

The device 1 comprises a hollow body 2 provided with a cylindrical-shaped side wall 3 of axis A and two base walls 4, 5. The body 2 also comprises an inlet opening 6 formed at the centre of the base wall 4 and communicating with an inlet conduit 7, and an outlet opening 8, communicating with an outlet conduit 9 and formed at the centre of base wall 5.

The device 1 comprises a helical-shaped deflector 10 coaxial to the hollow body 2 and housed therein, the outer edge of which is tangential to an inner surface 12 of the side wall 3 of the hollow body 2 so as to cooperate substantially sealingly with it.

Preferably, the deflector 10 is in the form of a ruled helicoid. In the example illustrated, the deflector 10 is in the form of a right helix with radial generatrices extending from the axis A to the inner surface 12 of the side wall 3.

The deflector 10 therefore forms a helical conduit 13 with the side wall 3 of the hollow body 2.

Conveniently, the helix pitch is chosen so that a section of the conduit 13 has an area approximately equal to that of the inlet opening 6, while its length, measured along a helical axis of the conduit, is between 5 and 10 times the axial length of the hollow body 2.

Finally, the device 1 comprises one or more sources 15 of UV-C radiation, preferably of a wavelength in the 250-280 nm field.

The sources 15 preferably consist of LEDS.

In the example illustrated, the sources 15 are arranged on the inner surface 12 of the side wall 3, e.g. along an axial generatrix of that surface, and equally spaced from each other by a distance equal to the pitch of the helicoid, so that each source is placed at an intermediate position between two successive turns of the helicoid.

Alternatively, the sources 15 may be arranged as alternate on opposite generatrices of the surface 12, or along a helical pathway extending along the helical conduit 13.

The sources 15 can be supplied by a battery 16, conveniently incorporated in the device 1.

The inner surface 12 of the side wall 3, as well as the inner surfaces of the base walls 4, 5 not affected by the openings 6, 8, are reflective (specular or diffuse). For the purposes of the present invention, the term “reflective” is used to denote a surface or coating having a reflectance of at least 0.8 (i.e. 80%).

For example, such surfaces may be defined by an aluminium film with a reflectance p=0.92.

Conveniently, the deflector 10 also has a reflective surface.

Thus, the hollow body 2, due to its total inner reflective surface area, behaves as an optical cavity that generates a multiplicative effect of illumination intensity due to the multiple reflections of radiation therein. The deflector 10, if made of reflective material, also contributes to the multiplication of intensity due to multiple reflections.

Such a multiplicative effect is as greater as higher the reflectance of the inner coating and deflector 10. Other materials suitable for constituting a reflective coating may be metal reflective covers (with a specular or diffuse surface) deposited under vacuum or by other methods, or vacuum-deposited reflective covers with a multilayer structure containing dielectric materials (e.g. SiO₂) to achieve high reflectivity in the UV-C region while filtering other bands and protecting the surface against reflectivity losses (such as through oxidation). The hollow body 2 may also consist of a metal sheet (e.g. aluminium) with a suitably processed reflective (specular or diffuse) inner surface.

The air contaminated volume has a very low UV radiation absorption, in the order of magnitude of 18, and therefore does not reduce the luminous intensity to any significant extent.

At flow speeds typically associated with a high-flow application such as for a hyperventilation helmet, of the order of magnitude of about 5 m/s, air maintains a substantially laminar flow. The residence time of the air inside the device 1, assuming that the speed remains unaltered compared to a comparative device without deflector 10, increases by a factor of between 5 and 10. Therefore, with the same irradiation energy density, the radiation dosage on the flow and consequently the disinfection effectiveness increases proportionally.

In FIG. 2 a non-invasive assisted ventilation system is overall illustrated by 20.

The system essentially comprises, in a known way:
a ventilator device 21, aiming at producing an air flow or possibly a gaseous mixture of oxygen-enriched air (hereinafter “air” for brevity's sake);
an air-conditioning device 22 aiming at controlling temperature and humidity; and
a patient/ventilator interface device 23, aiming at conveying air to the patient's external airway.

The interface device 23 may be represented, for example, by a ventilation helmet. The type of interface depends essentially on the air flow administered.

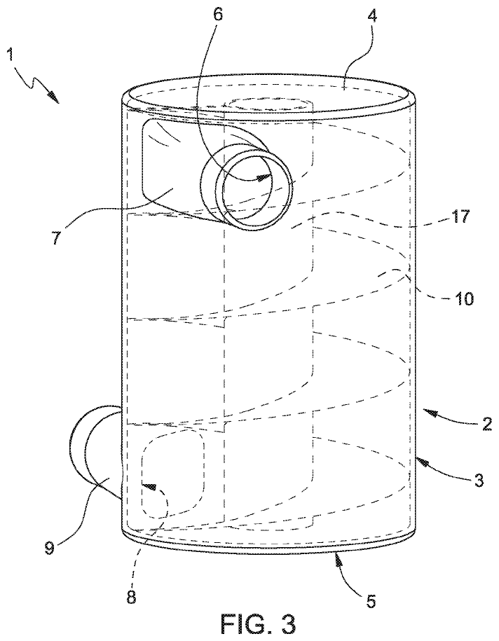

As schematised in FIG. 2, in some cases and predominantly in high-flow systems (e.g. in ventilation helmets used in hospitals), the exhaled air is channelled into an emission conduit 25, communicating with the device 1 made according to the invention. Therefore, the contaminated air, contained in the emission conduit 25, is passed through the disinfection device 1 before being reintroduced into the environment via the outlet conduit 9. In FIG. 3 a variant of the disinfection device of FIG. 1 is illustrated wherein the inlet 7 and outlet conduits 9 are made on the side wall 3 of the body 2, close to the respective base walls 4, 5. The conduits 7, 9 extend along respective extensions of the helical conduit 13, i.e. in a substantially tangential direction, so as not to cause abrupt changes in speed or pressure of the flow entering and leaving the device.

After examining the characteristics of the device 1 made according to the present invention, the advantages it allows to obtain are clear.

The increase in the residence time of the air in the device 1 due to the extension of its path along a helical trajectory imposed by the deflector 10 enables to effectively disinfect high-flow laminar air via multiple reflections of UV-C radiation within the device 1. Therefore, the device 1 made according to the present invention may be implemented in ventilation systems using high-flow ventilator/patient interfaces, such as in the solution of FIG. 2.

It is finally clear that modifications and variations can be made to the illustrated device 1 without thereby departing from the scope of protection defined by the claims.

1. A device for disinfecting an air flow, comprising a hollow body having an axis, a side wall and two base walls provided with internal reflective surfaces, an inlet opening and an outlet opening formed in, or near, the respective base walls, at least one source of UV-C radiation disposed within the hollow body, and a helical-shaped deflector axially housed in the hollow body;
wherein the hollow body is cylindrical and that an outer edge of the deflector cooperates substantially sealingly with an inner surface of the side wall of the hollow body so as to define with said side wall a helical conduit traversed by the entire flow.

2. Device as claimed in claim 1, wherein a cross-section of the helical conduit is approximately equal to that of the inlet opening of the hollow body.

3. Device as claimed in claim 1, comprising an inlet conduit and an outlet conduit arranged along respective extensions of the helical conduit.

4. Device as claimed in claim 1, wherein the helical conduit has a length of 5 to 10 times the axial length of the hollow body.

5. Device as claimed in claim 1, wherein the deflector is in the form of a ruled helicoid.

6. Device as claimed in claim 1, wherein the deflector is in the shape of a right helicoid.

7. Device as claimed in claim 1, wherein the deflector has a reflective surface.

8. Device as claimed in claim 7, wherein the inner surfaces of the hollow body and the surface of the deflector have a reflectance of at least 0.8.

9. Device as claimed in claim 8, wherein at least one of said surfaces has a coating comprising an aluminium film.

10. Device as claimed in claim 8, characterized in that at least one of said surfaces comprises a material optically specular in the UV-C band.

11. Device as claimed in claim 1, wherein the source of UV-C radiation comprises one or more LEDs arranged on the inner surface of the side wall of the hollow body.

12. An assisted ventilation system comprising a ventilator, a conditioning device, an interface device for conveying a flow produced by the ventilator to the external airway of the patient, and a disinfection device as claimed in claim 1, connected to the interface device via an emission conduit so as to intercept the air exhaled by the patient.
